(19) United States
(12) Patent Application Publication
(10) Pub. No.: US 2007/0260296 A1
(43) Pub. Date: Nov. 8, 2007

(54) Apparatus for Photodynamic Therapy

(76) Inventors: William Porter, County Cork (IE); Margaret Ayer Porter, County Cork (IE); Alexander Ovchinnikov, County Cork (IE)

Correspondence Address: DORSEY & WHITNEY LLP;INTELLECTUAL PROPERTY DEPARTMENT, SUITE 1500, 50 SOUTH SIXTH STREET, MINNEAPOLIS, MN, 55402-1498, US

(21) Appl. No.: 11/684,909

(22) Filed: Mar. 12, 2007

(30) Foreign Application Priority Data
Sep. 13, 2004 (IE) PCT/IE04/00118

(51) Int. Cl. A61N 5/06

(52) U.S. Cl. 607/88

(57) ABSTRACT

A therapeutic bed 1 for photodynamic therapy has a ground engaging base portion 2 with a patient support platform 3. A cover 4 is hingedly mounted on this base portion 2 and cooperates with the base portion 2 to form a patient treatment compartment 6 within which a patient 7 is fully bathed in light from LED panels 8 arranged about the patient support platform 3 to direct light of a preselected wavelength at the patient 7. The wavelength of the light corresponds to the peaks of absorption of an associated photosensitive agent administered to the patient 7 which has an affinity for malignant cells.

### CROSS REFERENCE TO RELATED APPLICATION(s)

This application is a continuation of International application number PCT/IE2004/000118, filed Sep. 13, 2004, the contents of which is incorporated herein by reference in its entirety.

This invention relates to a therapeutic apparatus and system for the treatment of systemic diseases such as cancer.

### BACKGROUND OF THE INVENTION

The most extensively used modem therapies for cancer are surgery, chemotherapy (various types of DNA poisons), and radiotherapy in the form of ionizing radiation; yet with few exceptions these are ineffectual against disseminated tumour and have extensive side effects. Surgery entails a long list of negatives including possible life threatening general anaesthesia, long recuperation periods, disruption of normal tissue, blood loss, infection, loss of function, pain and a list of other considerations too numerous to mention.

Chemotherapy also has a long and well known list of potentially life threatening side effects and additionally is poorly effective in most types of solid tumours, especially where metastatic spread has taken place. Additionally, chemotherapy is itself carcinogenic.

Radiation too is carcinogenic as well as having the disadvantage of extensive potential collateral damage to vital normal structures and functions. It is non specific and has many long term and significant side effects.

Photodynamic therapy has been developed as a treatment for cancer. In photodynamic therapy a patient is administered a photo sensitive agent such as a porphyrin which has a particular affinity for tumours. This agent is subsequently activated with light to destroy the tumours.

Photodynamic therapy can be utilised before or after surgery, chemotherapy and/or ionizing radiation therapy. None of these other therapies are compromised by photodynamic therapy and unlike the latter treatment, can be repeated many times with no resistance, developing, minimal morbidity and better functional results. Excellent cosmetic outcome with photodynamic therapy makes it important in skin lesions and cancer of the head and—neck where preserving function and respecting delicate underlying structures is critical. Treatment of extensive-areas of pleura and peritoneum can likewise be treated with relative ease, unlike radiation, which would result in unacceptable damage to underlying tissues. The adjunctive use of photodynamic therapy at the time of surgical removal of a primary tumour may be valuable to aid in the elimination of residual microscopic metastases. Finally, innovative uses of interstitial light delivery (light is delivered by inserting fiberoptic bundles through needles directed into the tumour mass under image guidance) have allowed a subcutaneous tumour 60 cubic centimetres to be successfully treated with Photofrin. Aside from care to avoid major blood vessels, this minimally invasive treatment is applicable to most areas of the body.

Barrett's Esophagus and cervical dysphasia are examples of conditions associated with frequent progression to malignancy. Photodynamic therapy has been found in numerous studies to be a successful treatment—for high-grade dysphasia of the esophagus and Japan has already approved Photofrin for use in cervical dysphasia.

Generally speaking photodynamic therapy uses the approach of direct illumination of a localised and identifiable tumour mass or condition. For internal tumours the application is with direct endoscopic visualisation and with the use of visually guided laser.

However, the vast majority of life threatening tumours and cancer are discovered at the stage when they have spread either regionally to lymph nodes or widely in the lymph system or circulatory system and metastasized to distant structures and tissues. As such the toxic forms of traditional therapies (chemo and radiation) or surgery are poorly effective or have in themselves such toxic effects on the body that there are severe limitations on their continued use.

Likewise, photodynamic therapy in the traditional sense also has limitations because the illumination and subsequent activation of the photosensitiser is applied to a finite lesion or area of tissue. Unfortunately the known photosensitive agents used in photodynamic therapy also have an affinity for normal cells such as skin tissue. Thus the right treatments are usually specifically targeted at the known tumour areas to avoid damage to skin and normal tissue. Also, a patient must avoid sunlight for a number of weeks after treatment or suffer severe sunburn or skin rashes. This is obviously considerably inconvenient for the patient.

The present invention is directed towards providing an improved therapeutic apparatus and system which overcomes these problems.

### SUMMARY OF INVENTION

According to the invention there is provided a therapeutic apparatus for photodynamic therapy, including:
a patient treatment compartment for reception of a patient, said patient treatment compartment having a light source which is operable to fully bathe a patient's body with light of a preselected wavelength or wavelengths when said patient is within the patient treatment compartment.

Preferably the wavelength or wavelengths of light supplied by the light source correspond to the peaks of absorption of a photosensitive agent administered to the patient prior to use of the therapeutic apparatus.

In one embodiment of the invention the light source is operable to generate light having wavelengths in the range 630-710 nm.

In another embodiment the light source is operable to generate light having wavelengths in the range 658-688 nm.

In a further embodiment the light source is operable to generate light having a wavelength of 663 nm.

In another embodiment means is provided for sensing the amount of energy directed from the light source into the patient treatment compartment and associated switching means is provided to switch off the light source when said sensed energy level reaches a predetermined amount.

In another embodiment said means comprises at least one light sensor mounted within the patient treatment compartment and connected to a controller which regulates operation of the light source, said controller having means for determining the energy level input from the light source into the compartment.

Preferably a plurality of light sensors are mounted spaced-apart within the compartment.

In another embodiment the light source comprises a plurality of light emitting diodes.

Conveniently a number of light generating panels which are mounted within the patient treatment compartment.

In another embodiment the light generating panels are positioned about an inner wall of the patent treatment compartment and arranged to direct light from each light generating panel towards a central portion of the patient treatment compartment.

In a particularly preferred embodiment the therapeutic apparatus is a bed including a base portion with a patient support platform, a cover mounted on the base portion and movable between an open position and a closed position, said cover when in the closed position defining with the base portion the patient treatment compartment.

In another embodiment the patient support platform is mounted on the base portion to support a patient centrally within the patient treatment compartment

In another embodiment the patient support platform is formed of a translucent material. Preferably the patient support platform is formed of a transparent material. Conveniently the patent support platform comprises clear perspex material.

In another embodiment the cover is hingedly mounted on the base portion for movement between the dosed position and the open position.

In a further embodiment ram means is provided for controlling movement of the cover between a raised open position and a lowered closed position.

In another embodiment the base portion and cover are L-shaped, the base portion forming a bottom wall and rear wall of the patient treatment compartment and the cover forming a top wall and a front wall of the patient treatment compartment, an inner end of said top wall being hingedly connected to an upper end of the rear wall.

In another embodiment the bottom wall and top wall are V shaped, each wall having light generating panels mounted thereon for directing light towards a central portion of the patient treatment compartment.

In another embodiment the invention provides a medical treatment device for photodynamic therapy including a patient treatment compartment or enclosure having means for fully bathing a patient's body in electromagnetic radiation of a preselected wavelength or wavelengths when said patient is, in the compartment. Generally the electromagnetic radiation will have a wavelength in the range 400 nm to 1000 nm, preferably from 600 nm to 900 nm, more preferably from 620 nm to 820 nm and most preferably from 630 nm to 710 nm.

In another embodiment lamp means is provided extending about the patient treatment compartment, said lamp means operable to direct light towards a patient located within the patient treatment compartment in use.

In another embodiment the lamp means includes an array of light emitting diodes (LED's) using coherent and/or non-coherent light which are operable to emit light of a selected wavelength or wavelengths.

Conveniently the LED's using coherent and/or non-coherent light may be provided in a number of LED panels mounted about the patient treatment compartment.

In a further embodiment the device includes control means which is operably connected to the LED's to regulate operation of the LED's.

In another aspect there is provided a medical treatment system including administering a photosensitive agent to a patient and subsequently irradiating the whole body of the patient with electromagnetic radiation of a selected wavelength or wavelengths in a controlled manner to activate said photosensitive agent. Preferably, the electromagnetic radiation used has a wavelength or wavelengths which correspond to the peaks of absorption of the photosensitive agent used. Preferably the photosensitive agent used is PhotoFlora.

In another aspect the invention provides a home treatment unit or kit for a patient to continue a course of treatment in their own home after initial treatment on the therapeutic bed device. The home treatment unit would include a light panel of LEDs operable for emitting light of a wavelength to activate the photosensitising agent. Preferably the light panel incorporates a microchip which records treatment data such as the time and duration of light usage. Conveniently means may be provided for downloading this information either directly or indirectly to a remote monitoring station (for example using a wireless GPRS communication between the home treatment unit and the clinic) allowing monitoring of a patient to ensure compliance with a recommended treatment schedule.

### BRIEF DESCRIPTION OF THE DRAWINGS

The invention will be more dearly understood by the following description of some embodiments thereof, given by way of example only, with reference to the accompanying drawings, in which;

FIG. 1 is a schematic sectional elevational view of a therapeutic bed according to the invention;

FIG. 2 is a sectional end elevational view of the therapeutic bed;

FIG. 3 is a view similar to FIG. 2 showing a cover of the bed in an open position;

FIG. 4 is an end elevational view of the bed; and

FIG. 5 is a perspective view of a light panel forming portion of a home treatment unit according to another aspect of the invention.

### DETAILED DESCRIPTION OF PREFERRED EMBODIMENTS

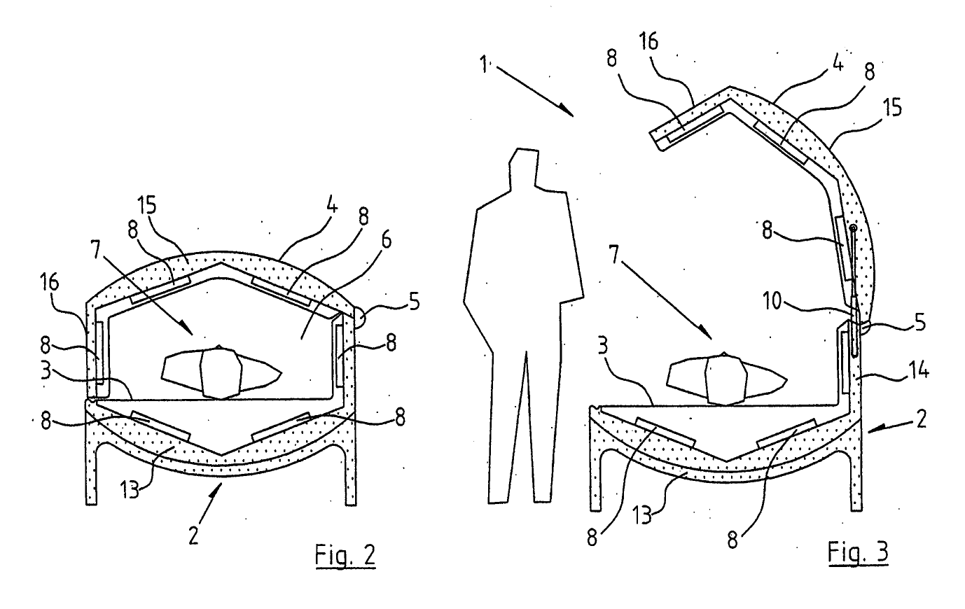

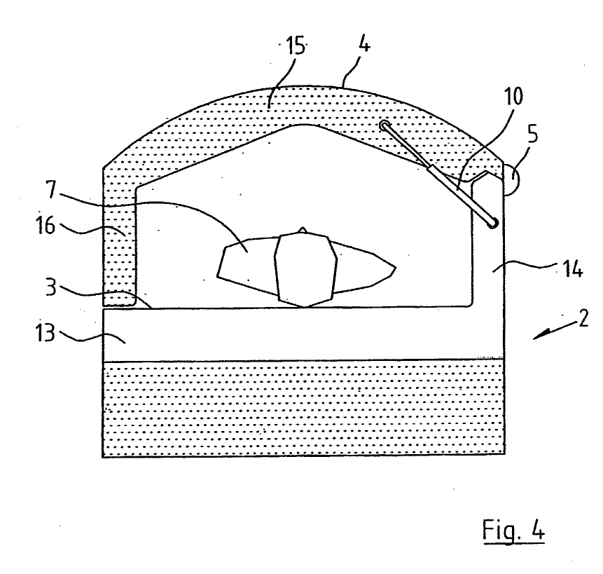

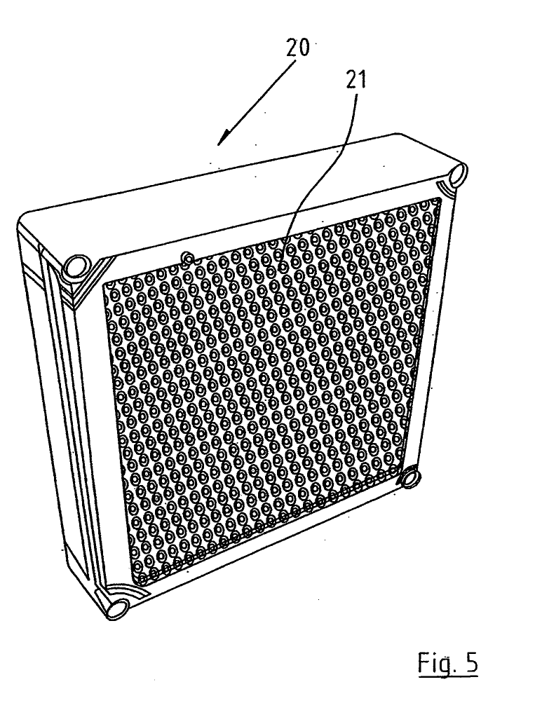

Referring to the drawings, there is illustrated a therapeutic bed according to the invention indicated generally by the reference numeral 1. The bed 1 has a ground engaging base portion 2 with a patient support platform 3. A cover 4 associated with the base portion 2 is hingedly mounted by a hinge 5 at one side of the base portion 2. The cover 4 when in a closed position (FIG. 2) defines with the base portion 2 a patient treatment compartment 6 within which a patient 7 lying on the patient support platform 3 can be subjected to a full body light treatment from an array of LED panels 8 arranged about the patient support platform 3 to direct light of a preselected wave length at the patient 7.

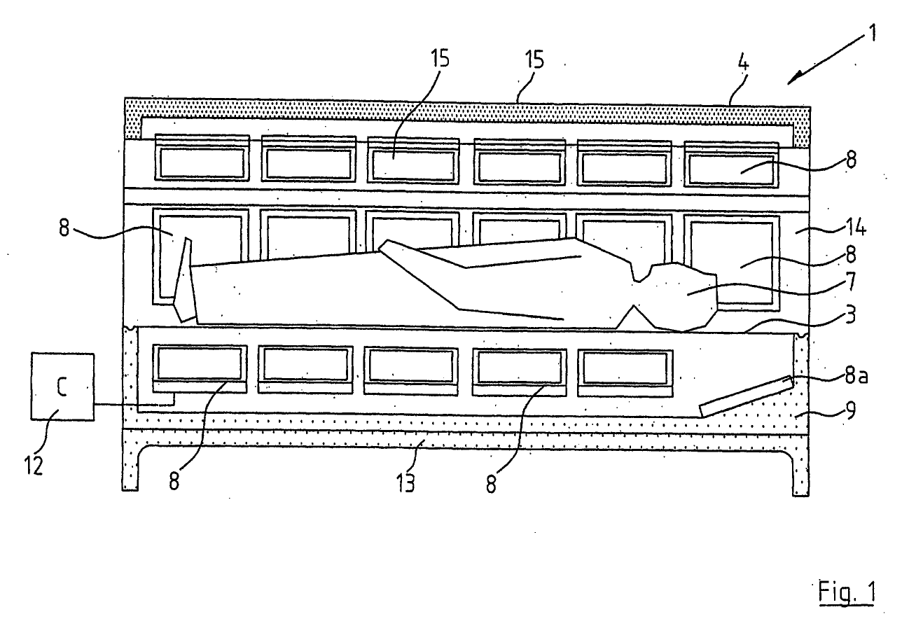

It will be noted that the inside faces of the base portion 2 and cover 4 upon which the LED panels 8 are mounted are sloped to direct light from the LED panels 8 inwardly towards a central portion of the compartment 6 above the patient support platform 3 on which the patient 7 lies. It wilt be noted also from FIG. 1 that at a head end 9 of the base portion 2 a number of LED panels 8a are mounted in an inclined position to direct fight rearwardly and upwardly towards the patients head. The arrangement of the LED panels 8 is such as to encircle the patient 7 bathing the full body of the patient 7 in light.

The patient support platform 3 is formed by a clear perspex panel to allow full transmission of light from the LED panels 8 through the patient support platform 3 to the patient 7.

A controller 12 incorporated in the bed 1 is operable to control the operation of the LED panels 8. Date relating to the patient such as name, type of cancer, stage, height and weight as well as the type, lot number and amount of photosensitises used can be recorded in the controller 12. A determination of the total amount of fight energy in joules required will be automatically calculated by the controller 12. Sensors (not shown) placed in appropriate locations about the bed 1 and connected to the controller 12 measure the light intensity levels a patient receives. An associated chronometer enables the overall amount of light in joules delivered in the course of a treatment on the bed 1 to be calculated. When the amount of joules of light equals the predetermined amount required for optimal treatment the controller 12 switches off the LED panels 8.

Provision for remotely accessing patient details and treatment parameters may be incorporated into the controller 12 in order that a central location or remote monitoring station can collate data for ongoing clinical studies.

Pneumatic arms 10 at each end of the bed 1 extending between the base portion and cover 4 adjacent the hinge 5 facilitate the controlled opening and closing of the cover 4 on the base portion 2.

The base portion 2 and cover 4 of the bed 1 are generally L-shaped in cross-section as can be seen in FIGS. 2 and 3. The base portion 2 forms a bottom wall 13 and an upstanding rear wall 14 of the compartment 6. The cover 4 forms a top wall 15 and a front wall 16 of the compartment 6. Further, at least inside faces of the bottom wall 13 and top wall 15 are V-shaped. LED panels 8 are mounted on an inside of each wall 13, to form a hexagonal array about the patient 7 as best seen in FIG. 2.

In use, the patient 7 is administered an amount of a photosensitising agent which will attach to tumours to which it has an affinity. The photosensitive agent may conveniently be administered orally to the patient. The patient 7 subsequently lies on the patient support platform 3 with the cover 4 closed. The controller operates the LED panels 8 to direct light associated with the photosensitising agent about the whole body of the patient 7 to activate the agent for attack and elimination of tumours to which the photosensitising agent is attached. The agent collects selectively in cancer tissue and when exposed to light becomes activated, releasing a highly energised free radical form of oxygen known as singlet oxygen. Singlet oxygen destroys cancer cells from the inside out, while leaving normal tissues largely unaffected. When the patient has received the pre-calculated required amount of fight energy the controller switches off the LED's.

Photosensitisers used are activated in the higher wavelengths, typically in the order of 600 nm-900+ nm which-allow for deeper penetration of light into the body. The bed I includes LED's that emit a light in the specific wavelengths that correspond to the exact peaks of absorption of the specific photosensitiser used. This assures maximum activation of the photosensitiser as well as the application of higher wavelengths that penetrate deeply into the body from maximum efficacy of tumour destruction even in the middle of the body. Because the bed 1 floods the entire body with light which is able to penetrate deeply into the body this addresses the problem of treating the widespread metastatic spread of tumour.

It is also envisaged that the treatment bed may be used in combination with photosensitive agents for the treatment of other systemic diseases such as atherosclerotic disease, rheumatoid and inflammatory arthritis for example. Further, it may also be possible to treat bacterial, viral and fungal infections using the system and apparatus of the invention. Also treatment off psoriasis, acne, alopecia areata and portwine stains and hair removal may also be possible. Likewise, the treatment may be useful as a prophylactic therapy for the reduction or elimination of subclinical or micrometastases. Individuals with genetic occupational or lifestyle behaviours (smokers) that predispose them to cancer/infectious diseases etc. could possibly benefit from therapy in this apparatus.

It will be appreciated that the system and apparatus of the invention allows treatment of the whole body of the patient. The photosensitive agent is highly selective and attaches to abnormal cells which are eliminated upon subjection of the patient to the light treatment. Normal tissue and structures are not adversely affected. The arrangement of the bed gives comprehensive coverage of the entire body to ensure the greatest possibility of successful activation of photosensitiser molecules that are absorbed on tumour or diseased tissue. The system allows outpatient treatment with few side effects and minimal disruption of the patients life.

While a bed has been described herein for the full body light treatment other arrangements are possible such as a compartment in which the patient stands up or sits down during the light treatment. What is important is that the fall body of the patient is bathed in fight of the desired wavelength.

A particularly suitable photosensitive agent for use together with the bed is PhotoFlora which is derived from microscopic chlorophyll containing plants such as Spirulia. PhotoFlora is activated at higher light wavelengths which allows a greater depth of penetration and a higher yield of singlet oxygen. It is very cancer-specific, accumulating preferentially in cancer tissue and is quickly cleared from the body. Typically the PhotoFlora is 95% metabolised and cleared from the body within 24 hours. PhotoFlora is a chlorin derivative with an intensive absorption band at 663 nm. It also has an intense flourescence reaction on tumours in the range of 658 nm to 688 nm.

Referring now to FIG. 5 there is shown a light panel assembly indicated generally by the reference numeral 20 forming portion of a home treatment unit which a patient may use to continue a course of treatment in their own home over a period of time after initial treatment on the therapeutic bed described previously. The light panel 20 includes an array of LEDs 21 which similarly to those described in the bed previously emit light of a wavelength to activate the associated photosensitising agent. The home treatment kit will also include a supply of the photosensitising agent for the patient to take over the extended treatment period for co-operation with the light panel 20 in treating the disease. The light panel 20 incorporates a microchip which records treatment data such as the time and duration of the light usage facilitating monitoring of patient compliance with a recommended treatment schedule. This data may be transferred by USB-etc. or with the increasing usage of wireless technology in the form of IR, Blue Tooth, Wi-Fi etc. to a computer, laptop or handheld device and then transferred on to a remote monitoring station at a treatment clinic for example. Thus medical personnel at the treatment clinic can monitor the treatment progress.

In use, the patient haven taken the required quantity of photosensitising agent will operate the light panel 20 directing right from the LEDs 21 at whichever part of their body requires the treatment.

The invention is not limited to the embodiments hereinbefore described which may be varied in both construction and detail within the scope of the appended loans.

What is claimed is:

1. A therapeutic apparatus for photodynamic therapy, including:
a patient treatment compartment for reception of a patient, said patient treatment compartment having a light source which is operable to fully bathe a patient's body with light of a preselected wavelength or wavelengths when said patient is within the patient treatment compartment.

2. The apparatus as claimed in claim 1, wherein the light source is operable to generate light having wavelengths in the range 630-71 nm.

3. The apparatus as claimed in claim 2, wherein the light source is operable to generate light having wavelengths in the range 658-688 nm.

4. The apparatus as claimed in claim 3, wherein the light source is operable to generate light having a wavelength of 663 nm.

5. The apparatus as claimed in 1, wherein a detector is provided for measuring the amount of energy directed from the light source into the patient treatment compartment and associated mechanism is provided to switch off the light source when said sensed energy level reaches a predetermined amount.

6. The apparatus as claimed in claim 5, wherein said detector comprises at least one light sensor mounted within the patient treatment compartment and connected to a controller which regulates operation of the light source, wherein said controller monitors the energy level input from the light source into the compartment.

7. The apparatus as claimed in claim 6, wherein a plurality of light sensors are mounted about the patient treatment compartment.

8. The apparatus as claimed in claim 1, wherein the light source comprises a plurality of light emitting diodes.

9. The apparatus as claimed in claim 8, wherein light emitting diodes are provided on a number of light generating panels which are mounted within the patient treatment compartment.

10. The apparatus as claimed in claim 9, wherein said light generating panels are positioned about an inner wall of the patent treatment compartment and arranged to direct light from each light generating panel towards a central portion of the patient treatment compartment.

11. The apparatus as claimed in claim 1, wherein the therapeutic apparatus comprises a bed including a base portion with a patient support platform, a cover mounted on the base portion and movable between an open position and a closed position, said cover when in the closed position defining with the base portion the patient treatment compartment.

12. The apparatus as claimed in claim 11, wherein the patient support platform is mounted on the base portion to support a patient centrally within the patent treatment compartment.

13. The apparatus as claimed in claim 11, wherein the patient support platform is formed of a translucent material to allow through passage of light from a portion of the light source located beneath the patient support platform.

14. The apparatus as claimed in claim 13, wherein the patient support platform is formed of a transparent material.

15. The apparatus as claimed in claim 14, wherein the patient support platform comprises a clear perspex material.

16. The apparatus as claimed in claim 11, wherein the cover is hingedly mounted on the base portion for movement between the closed position and the open position.

17. The apparatus as claimed in claim 16, wherein pneumatic arms are provided for controlling movement of the cover between a raised open position and a lowered closed position.

18. The apparatus as claimed in claim 11, wherein the base portion and cover are L-shaped, the base portion forming a bottom wall and rear wall of the patient treatment compartment and the cover forming a top wall and a front wall of the patient treatment compartment, an inner end of said top wall being hingedly connected to an upper end of the rear wall.

19. The apparatus as claimed in claim 18, wherein the bottom wall and top wall are V-shaped, each wall having light generating panels mounted thereon for directing light towards a central portion of the patient treatment compartment.

20. The apparatus as claimed in claim 19, wherein a plurality of light generating panels are arranged in a hexagonal array within the patient treatment compartment.

21. A photodynamic method comprising the steps:
administering a photosensitive agent to a patient; and
illuminating the whole body of the patient via the therapeutic apparatus as claimed in claim 1 to activate said photosensitive agent.

22. The therapeutic method as claimed in claim 21, wherein the light comprises a wavelength or wavelengths which correspond to the peaks of absorption of the photosensitive agent.

23. The therapeutic method as claimed in claim 22, wherein the photosensitive agent is photoflora.
